(19) United States
(12) Patent Application Publication
(10) Pub. No.: US 2026/0137739 A1
(43) Pub. Date: May 21, 2026

(54) NOVEL BACTERIOPHAGE COMPOSITIONS, FORMULATIONS AND METHODS OF MAKING AND USE

(71) Applicant: Purdue Research Foundation, West Lafayette, IN (US)

(72) Inventors: Qi Zhou, West Lafayette, IN (US); Vaibhav Pathak, West Lafayette, IN (US); Li Qu, West Lafayette, IN (US)

(73) Assignee: Purdue Research Foundation, West Lafayette, IN (US)

(21) Appl. No.: 19/392,867

(22) Filed: Nov. 18, 2025

Related U.S. Application Data
(60) Provisional application No. 63/723,457, filed on Nov. 21, 2024.

(51) Int. Cl. A61K 35/76; A61K 9/16; C12N 7/00

(52) U.S. Cl. CPC A61K 35/76; A61K 9/1617; A61K 9/1658; A61K 9/1682; C12N 7/00; C12N 2795/00032

(57) ABSTRACT

The present disclosure provides for novel compositions and pharmaceutical formulations of bacteriophage suitable for stable storage and effective therapeutic treatment of a patient.

### REFERENCE TO RELATED APPLICATIONS

This present patent application relates to and claims the priority under 35 U.S.C. § 119 (e) to U.S. Provisional Patent Application Ser. No. 62/723,457 filed on Nov. 21, 2024, the content of which is hereby incorporated by reference in its entirety into the present disclosure.

### STATEMENT OF GOVERNMENT SUPPORT

This invention was made with government support under grant AI146160 and HL167828 awarded by the National Institutes of Health. The government has certain rights in the invention.

### TECHNICAL FIELD

The present disclosure generally relates to the area of powdered compositions of bacteriophage, pharmaceutical formulations thereof, methods of making, and methods of use. The present disclosure describes the development of bacteriophage powder compositions which demonstrate both stability and suitability for use in pharmaceutical formulations. The present disclosure describes powder compositions made utilizing a combination of excipients. In particular, the present disclosure describes spray dried compositions of bacteriophage in combination with gelatin and an amino acid such as leucine, and their use in pharmaceutical formulations.

### BACKGROUND

This section introduces aspects that may help facilitate a better understanding of the disclosure. Accordingly, these statements are to be read in this light and are not to be understood as admissions about what is or is not prior art.

Antibiotic resistance is rapidly becoming a global threat to human health and modern medicine. In 2019, antibiotic resistance was linked to an estimated 1.2 million deaths worldwide. This problem has been exacerbated by reduced interest of industries in antibiotic development and a decline in the number of new approved antibiotics in recent decades. Bacteriophages are potent antibacterial agents that can replace or complement antibiotics. These are naturally occurring viruses that infect bacteria. Bacteriophages that are obligately “lytic” self-replicate within bacterial cells and release progeny phages by cell lysis. Phages have several advantages over antibiotics: a high specificity to target bacteria, the ability to multiply in the presence of bacteria, and the ability to evolve along with the host bacteria.

Antibiotic-resistant pathogens commonly infect the respiratory system. Among antibiotic-resistant infections, lower respiratory and thorax infections cause the largest burden of infections in 2019; globally, these infections caused over 400,000 deaths that were linked to resistance. Therefore, the application of phages is highly relevant for treating multidrug resistant lung infections.

Several studies using animal infection models have demonstrated the efficacy of phages. Semler et al. (Aerosol phage therapy efficacy in Burkholderia cepacian complex respiratory infections, Antimicrob Agents Chemother, 2014 July; 58 (7): 4005-13) showed that aerosolized phage delivery (Myoviridae KS12) to mice lungs resulted in a 2-log reduction in the median bacterial load of acute Burkholderia cenocepacia K56-2 lung infection. Yang et al. (Therapeutic effect of the YH6 phage in a murine hemorrhagic pneumonia model, Research in Microbiology October 2015, Vol. 166 (8): 633-643) demonstrated complete eradication of P. aeruginosa D9 cells in a murine hemorrhagic pneumonia model; a single dose of an N4-like phage (YH6) with an MOI (phage-to-bacteria ratio) of 0.1 was intranasally administered 2 h after infection and resulted in 100% survival.

Administration of antibiotics through the pulmonary route can deliver a significantly higher dose to the lungs than systemic administration (Borghardt, Kloft, & Sharma, Inhaled Therapy in Respiratory Disease: The Complex Interplay of Pulmonary Kinetic Processes, Can Respir J., June 19; 2018:2732017). Dry powder inhalers (DPIs) are a convenient dosage form for pulmonary delivery due to their ease of use and portability. They also show superior chemical stability than solutions- or suspensions-based dosage forms and can deliver a high drug dose. Formulation excipients are critical to preserving the activity of phages from desiccation stress during the preparation of dry powder formulations and their subsequent storage. Disaccharides such as trehalose, sucrose, and lactose are commonly used as lyoprotectant excipients for phages and proteins (Chang et al., 2017 Production of highly stable spray dried phage formulations for treatment of Pseudomonas aeruginosa lung infection. European Journal of Pharmaceutics and Biopharmaceutics, 121, 1-13; Leung, S. S. Y., et al. 2017 Effects of storage conditions on the stability of spray dried, inhalable bacteriophage powders. International journal of pharmaceutics, 521(1), 141-149)

Nebulizer formulation of two proteins containing gelatin showed better activity retention after nebulization (Li et al., 2022 Gelatin Stabilizes Nebulized Proteins in Pulmonary Drug Delivery against COVID-19. ACS Biomaterials Science & Engineering, 8(6), 2553-2563.). Thus, we wish to test gelatin as a phage stabilizer in dry powders. In addition to high phage stability, dry powders should also have good aerosolization because it allows efficient drug delivery to the lungs. In this study, we aimed to formulate inhalable powders of a Pseudomonas aeruginosa phage using spray drying as the manufacturing technique. We studied the effect of six excipients on phage stability during preparation, and aerosolization: trehalose, lactose, gelatin, hydrolyzed gelatin, mannitol and leucine. Work on nanostructures was described by Langecker et al. (Synthetic Lipid Membrane Channels Formed by Designed DNA Nanostructures, Science, Vol. 338 No. 6109, 16 Nov. 2012, pp 932-936). A review of DNA nanotechnology is provided by Seeman & Sleiman (DNA nanotechnology, Nature Reviews/Materials Vol. 3, Article Number 17068, published online 8 Nov. 2017). Xing et al. (Highly shape- and size-tunable membrane nanopores made with DNA, Nature Nanotechnology, Vol. 17, July 2022, 708-712) describe work on shaping nanopore structures.

An example of co-spray drying is described in U.S. Pat. No. 11,110,085 B2 (Qi Zhou and Nivedita Shetty, Co-spray drying of ciprofloxacin and colistin and the uses thereof.

The present disclosure addresses the problems of stability and availability of bacteriophage of therapeutic administration by providing methods for making and compositions which provide for better stability and availability of bacteriophage which is useful for therapeutic administration and other uses.

Here we describe results that describe the development of Pseudomonas aeruginosa bacteriophage 95 (ATCC 14211-B1) as a stable and aerosolizable dry powder formulation. We specifically investigated the effects of different excipients (e.g. trehalose, lactose, leucine, mannitol, gelatin and hydrolyzed gelatin) on the aerosol performance and stabilization of spray dried bacteriophage powder formulations. Our data demonstrated that hydrolyzed gelatin was the most effective bacteriophage stabilizer during spray drying, showing a titer reduction of only 0.6 log. When stored at 4° C., hydrolyzed gelatin and gelatin showed the best stability with negligible bacteriophage titer reduction for 12 weeks. The in vitro aerosol performance of spray dried formulations was assessed using a multistage liquid impinger coupled with a low-resistance RS01 inhaler device. The hydrolyzed gelatin formulation showed an emitted dose (ED) of 60% and fine particle fraction (FPF) of 77%. The inclusion of leucine and trileucine at 5% w/w significantly improved the aerosol performance of the hydrolyzed gelatin-bacteriophage formulation to an ED of 90% and an FPF of 80-86%; both were amorphous matrix that showed low bacteriophage titer reduction (0.7 log). This is the first study to report that a composite spray dried matrix of hydrolyzed gelatin (as a stabilizer) with leucine or trileucine (as dispersion enhancers) provides both high bacteriophage stability and excellent aerosol performance, making it a promising dry powder formulation for treating pulmonary bacterial infections.

### BRIEF SUMMARY

The present invention generally relates to powder compositions comprising a bacteriophage and at least two excipients, pharmaceutical formulations thereof and methods of making such.

The present invention teaches a method for manufacturing a powder composition consisting of an amino acid, gelatin and bacteriophage, comprising:
a. preparing a bacteriophage suspension;
b. prepare an excipient solution by dissolving an amino acid and gelatin, or a pharmaceutically acceptable salts thereof, in an aqueous or an organic solvent;
c. create a mixture by combining bacteriophage suspension to the excipient solution; and
d. spray-drying the mixture to create a powder composition.

The present invention teaches a method for manufacturing a powder composition consisting of an amino acid, gelatin and bacteriophage, comprising:
preparing a bacteriophage suspension;
prepare an excipient solution by dissolving an amino acid and gelatin, or a pharmaceutically acceptable salts thereof, in an aqueous or an organic solvent;
c. create a mixture by combining bacteriophage suspension to the excipient solution; and
d. spray-drying the mixture to create a powder composition;
   where the amino acid is selected from the group consisting of nonpolar, non aromatic amino acids glycine, alanine, valine, leucine and isoleucine.

The present invention teaches a method as described above, where the amino acid is leucine or a derivative, analog or mimetic thereof. In a further embodiment the present invention teaches where the leucine or derivative, analog or mimetic thereof is selected from the group L-leucine, iso-leucine, or tri-leucine.

The present invention teaches a method as described where the gelatin is a unhydrolyzed gelatin, hydrolyzed gelatin or other type of gelatin or gelatin analog.

Thus the present invention teaches a method for manufacturing a powder composition consisting of an amino acid, gelatin and bacteriophage, which results in a bacteriophage-excipient powder composition that provides for an inhalable fraction of bacteriophage with an increased stability and aerosolization stability as compared with bacteriophage alone.

The present invention teaches a method for making a shelf stable powder composition which comprises amino acid, gelatin and bacteriophage.

The present invention provides for a shelf stable powder composition made by the method as described above wherein the proportion of amino acid to gelatin is from about 5% amino acid to about 95% gelatin. The present invention provides for variation is such range which encompasses the presence of more amino acid such that the amount of amino acid is about 10% and the amount of gelatin is about 90%. In general, the present invention encompasses the use of gelatin with amino acid, where the amino acid content is from about 1% to 25% either as a single amino acid or a combination of two or more amino acids.

In addition, the present invention teaches where the gelatin analog is semi-synthetic gelatin analogs (gelatin mehacryloyl), agar-agar, carrageenan, alginates, cornstarch, pectin, Hypromellose and the like.

The present invention teaches a method for manufacturing a powder composition consisting of an amino acid, gelatin and bacteriophage, and for the making of a pharmaceutical formulation of bacteriophage comprising
a. dissolving amino acid and gelatin, or a pharmaceutically acceptable salt thereof, in an aqueous or an organic medium to prepare an excipient solution;
b. optionally adding one or more pharmaceutically acceptable excipients, carrier or diluent to the excipient solution;
c. preparing a bacteriophage suspension;
c. combining the bacteriophage suspension with the excipient solution to make a mixture; and
d. spray-drying the mixture to create a powder composition.

The method as described above, which results in a dried powder composition which contains bacteriophage where said powder composition has an improved inhalable fraction of bacteriophage and an increased stability and aerosolization stability as compared with bacteriophage alone.

In addition, the method of the invention encompasses where the gelatin is selected from the group consisting of unhydrolyzed gelatin, hydrolyzed gelatin or other type of gelatin or gelatin analog. In a further embodiment the method of the invention encompasses where the amino acid is selected from the group consisting of Leucine, L-leucine, iso-leucine, or tri-leucine

Thus the present invention provides for a bacteriophage powder composition made by the method as described above, consisting of an amino acid, gelatin and bacteriophage.

The present invention provides for a powder composition made by the method as described above wherein the proportion of amino acid to gelatin is from about 5% amino acid to about 95% gelatin. The present invention provides for variation is such range which encompasses the presence of more amino acid such that the amount of amino acid is about 10% and the amount of gelatin is about 90%. In general, the present invention encompasses the use of gelatin with amino acid, where the amino acid content is from about 1% to 25% either as a single amino acid or a combination of two or more amino acids.

In a further embodiment the present invention teaches a bacteriophage pharmaceutical composition, made by a method for manufacturing a powder composition consisting of an amino acid, gelatin and bacteriophage, with a pharmaceutically acceptable carrier, excipient or diluent.

The present invention encompasses the use of a composition made by the method of the invention for manufacturing a powder composition consisting of an amino acid, gelatin and bacteriophage, for the treatment of a bacterial disease in a patient in need thereof. It is further encompassed by the invention, the use of a composition as described for the treatment of a bacterial disease in a patient in need thereof.

It is further envisioned by the present invention, that the composition made by the method of the invention for manufacturing a powder composition consisting of an amino acid, gelatin and bacteriophage, to be used in a method for treating or prophylactically treating a patient with a bacterial infection comprising administering a therapeutic amount of a composition of claim 13 of 14 to a patient in need thereof. It is further envisioned that such method of treatment or prophylaxis may be accomplished where administration is in combination with or coordination with another therapeutic agent. It is further envisioned that the compounds, pharmaceutical formulations, and treatments can be fashioned where the administration of the therapeutic dose is via any medically appropriate route of administration and scaled appropriately.

Thus the present invention encompasses treating a mammal for infection with a bacteria pathogen. Such treatment encompasses the use of a pharmaceutical composition comprising a powder composition consisting of an amino acid, gelatin and bacteriophage.

Thus the present invention encompasses a method for treating a mammal for bacterial infection, said method comprising administering a therapeutic amount of a pharmaceutical composition comprising a powder composition consisting of an amino acid, gelatin and bacteriophage.

Thus the present invention encompasses methods for creating a composite spray dried matrix of hydrolyzed gelatin with leucine or trileucine which provides both high bacteriophage stability, and excellent aerosol performance as a dry powder formulation for treating bacterial infection. In one embodiment the bacteriophage is a Pseudomonas aeruginosa bacteriophage.

These and other features, aspects and advantages of the methods of the present disclosure will become better understood with reference to the following drawings, descriptions and claims.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

A better understanding of the present disclosure will be obtained upon reference to the following description in conjunction with the accompanying drawings.

FIG. 1 graphs Change in titer of Pa 95 phage in single-excipient spray dried formulations due to spray drying with respect to the feed solution

FIG. 2 depicts Powder X-ray diffraction pattern of spray dried single-excipient formulations of Pa 95 phage.

FIG. 3 shows photographs of representative scanning electron microscopy images of spray dried single-excipient formulations of Atomic Force Microscopy of the DNA nanocube structures.

FIG. 4A is a graph showing Percent recovery, percent emitted dose (ED) and percent fine particle fraction (FPF) of Pa 95 phage in spray dried single-excipient formulations. Error bars indicate standard deviation (n=3-4). FIG. 4B is a graph showing Recovery, emitted dose (ED), and fine particle fraction (FPF) of spray dried phage formulations based on MSLI analysis. Error bars indicate standard deviation (n=3 or 4). (*) indicates the value is significantly different (p<0.05) from that of hydrolyzed gelatin.

FIG. 5 shows a graph of Change in the Pa 95 phage titer in spray dried formulations of trehalose, lactose, gelatin and hydrolyzed gelatin during storage in closed vials at 4° C. Error bars indicate standard deviation (n=3-4).

FIGS. 6A and B are graphs which show slightly reordered presentation of data. Change in titer of Pa 95 phage in spray dried HG-leucine and HG-trileucine formulations due to spray drying with respect to the feed solution. Change in the phage titer of spray dried formulations containing hydrolyzed gelatin (HG) and dispersion enhancer (leucine or trileucine) (mean±SD, n=3). HG100 is HG.

FIG. 7 graphs Powder X-ray diffraction pattern of spray dried HG-leucine and HG-trileucine formulations of Pa 95 phage.

FIG. 8 shows photographs of Representative scanning electron microscopy images of spray dried particles of HG-leucine and HG-trileucine formulations.

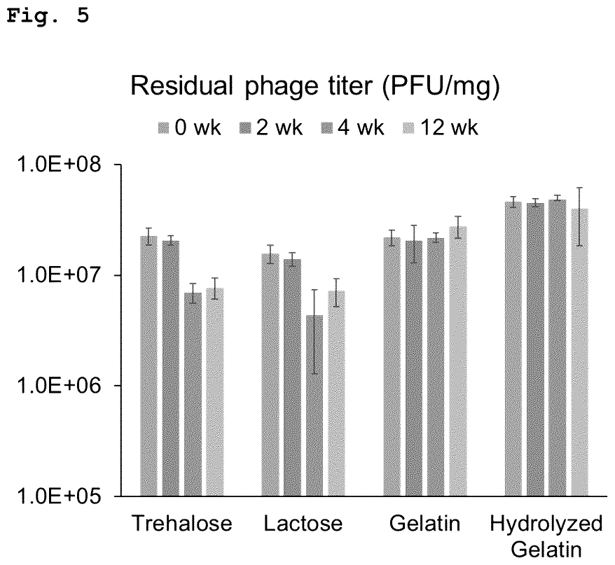

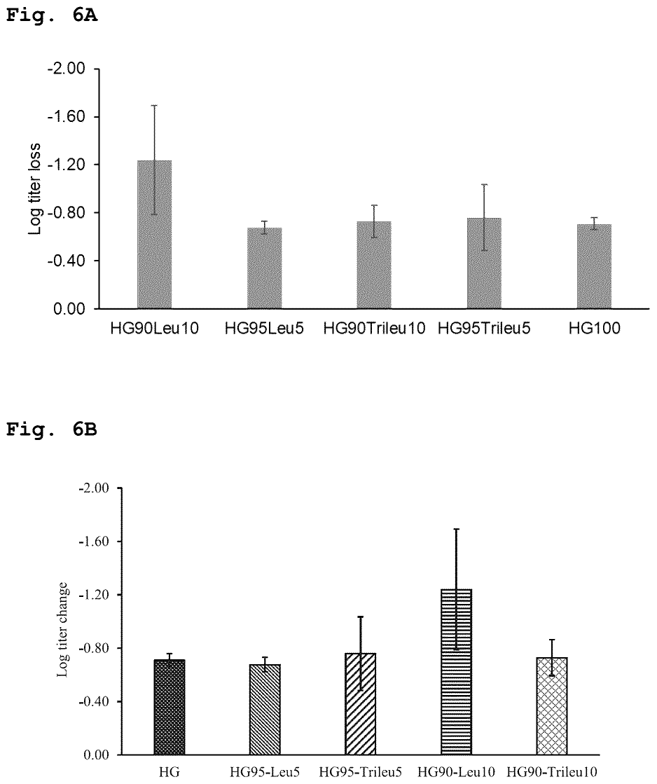

FIG. 9A-C shows graphs of Percent recovery, percent emitted dose (ED) and percent fine particle fraction (FPF) of Pa 95 phage in spray dried formulations containing HG with 10% leucine/trileucine (FIG. 9A) or 5% leucine/trileucine (FIG. 9B). Error bars indicate standard deviation (n=3). FIG. 9C graphically presents data showing recovery, emitted dose (ED) and fine particle fraction (FPF) of Pseudomonas aeruginosa phage 95 phage in spray dried hydrolyzed gelatin (HG) and 90:10 and
95:5 formulations of hydrolyzed gelatin with leucine or trileucine. Error bars indicate standard deviation (n=3). (*) indicates the value is significantly different (p<
0.05) from that of hydrolyzed gelatin.

FIG. 10 is a table of data for Particle size and glass transition temperature of spray formulations. Data are presented as mean±standard deviation (n=4).

FIG. 11 is a table of composition of formulations containing hydrolyzed gelatin (HG) and dispersion enhancers.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

Unless defined otherwise, the scientific and technology nomenclatures have the same meaning as commonly understood by a person in the ordinary skill in the art pertaining to this disclosure.

The term “patient” includes human and non-human animals such as companion animals (dogs and cats and the like) and livestock animals. Livestock animals are animals raised for food production. The patient to be treated is preferably a mammal, in particular a human being.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffered solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

It is to be understood that the total daily usage of the compounds and compositions described herein may be decided by the attending physician within the scope of sound medical judgment. The specific therapeutically effective dose level for any particular patient will depend upon a variety of factors, including the disorder being treated and the severity of the disorder; activity of the specific compound employed; the specific composition employed; the age, body weight, general health, gender, and diet of the patient: the time of administration, and rate of excretion of the specific compound employed, the duration of the treatment, the drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosage may be single or divided, and may be administered according to a wide variety of dosing protocols, including q.d., b.i.d., t.i.d., or even every other day, once a week, once a month, and the like. In each case the therapeutically effective amount described herein corresponds to the instance of administration, or alternatively to the total daily, weekly, or monthly dose.

As used herein, the term “therapeutically effective amount” refers to that amount of active compound or pharmaceutical agent that elicits the biological or medicinal response in a tissue system, animal or human that is being sought by a researcher, veterinarian, medical doctor or other clinicians, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment.

As used herein, the term “therapeutically effective amount” refers to the amount to be administered to a patient, and may be based on body surface area, patient weight, and/or patient condition. In addition, it is appreciated that there is an interrelationship of dosages determined for humans and those dosages determined for animals, including test animals (illustratively based on milligrams per meter squared of body surface) as described by Freireich, E. J., et al., Cancer Chemother. Rep. 1966, 50 (4), 219, the disclosure of which is incorporated herein by reference. Body surface area may be approximately determined from patient height and weight (see, e.g., Scientific Tables, Geigy Pharmaceuticals, Ardley, New York, pages 537-538 (1970)). A therapeutically effective amount of the compounds described herein may be defined as any amount useful for inhibiting the growth of (or killing) a population of malignant cells or cancer cells, such as may be found in a patient in need of relief from such cancer or malignancy. Typically, such effective amounts range from about 5 mg/kg to about 500 mg/kg, from about 5 mg/kg to about 250 mg/kg, and/or from about 5 mg/kg to about 150 mg/kg of compound per patient body weight. It is appreciated that effective doses may also vary depending on the route of administration, optional excipient usage, and the possibility of co-usage of the compound with other conventional and non-conventional therapeutic treatments, including other anti-tumor agents, radiation therapy, and the like.

This application provides for the development of bacteriophage, in particular Pseudomonas aeruginosa bacteriophage 95 (ATCC 14211-B1), as a stable and aerosolizable dry powder formulation. We specifically investigated the effects of different excipients (e.g. trehalose, lactose, leucine, mannitol, gelatin and hydrolyzed gelatin) on the aerosol performance and stabilization of spray dried bacteriophage powder formulations. Our data demonstrated that hydrolyzed gelatin was the most effective bacteriophage stabilizer during spray drying, showing a titer reduction of only 0.6 log. When stored at 4° C., hydrolyzed gelatin and gelatin showed the best stability with negligible bacteriophage titer reduction for 12 weeks providing for a shelf stable formulation. The in vitro aerosol performance of spray dried formulations was assessed using a multistage liquid impinger coupled with a low-resistance RS01 inhaler device. The hydrolyzed gelatin formulation showed an emitted dose (ED) of 60% and fine particle fraction (FPF) of 77%. The inclusion of leucine and trileucine at 5% w/w significantly improved the aerosol performance of the hydrolyzed gelatin-bacteriophage formulation to an ED of 90% and an FPF of 80-86%; both were amorphous matrix that showed low bacteriophage titer reduction (0.7 log). This is the first study to report that a composite spray dried matrix of hydrolyzed gelatin (as a stabilizer) with leucine or trileucine (as dispersion enhancers) provides both high bacteriophage stability and excellent aerosol performance, making it a promising dry powder formulation for treating pulmonary bacterial infections.

It was our belief that the preparation of a useful bacteriophage therapeutic providing for sufficient dosage and efficacy could be accomplished via preparation of a powdered composition. We decided that the key features for improved composition was to focus on both bacteriophage (phage) stability in drying. For an effective phage formulation for inhalation, it is important to have a high viable phage titer after preparation and storage. It is equally critical for the powder to efficiently deliver the viable phages to the lungs.

We discovered that the process of the present disclosure achieved this.

In particular, the present disclosure provides for a method for manufacturing a powder composition consisting of an amino acid, gelatin and bacteriophage, comprising the steps of
a. dissolving leucine and gelatin, or a pharmaceutically acceptable salts thereof, respectively, in an aqueous or an organic medium to prepare a solution;
b. adding one or more pharmaceutically acceptable excipients to said solution; and
c. combining bacteriophage suspension to the said solution; and
d. spray-drying to afford said powder composition with an improved inhalable fraction of said bacteriophage and an increased stability and aerosolization stability as compared with bacteriophage alone.

The present disclosure provides for the process described above, where the amino acid is leucine or a derivative, analog or mimetic thereof, such as L-leucine, iso-leucine, tri-leucine or other leucine type of amino acid. Thus the present disclosure provides for a method of manufacture as described above where the amino acid is selected from the group consisting of Leucine, L-leucine, iso-leucine, tri-leucine or leucine analog. In a preferred embodiment the amino acid is Leucine.

The present invention provides for a powder composition made by the method as described above wherein the excipient comprises a measured proportion of amino acid to gelatin from about 5 amino acid to about 95 gelatin. The present invention provides for variation is such range which encompasses the presence of more amino acid such that the amount of amino acid is about 10 and the amount of gelatin is about 90. In general, the present invention encompasses an excipient that comprises gelatin with amino acid, where the amino acid content is from about 1% to 25% either as a single amino acid or a combination of two or more amino acids by combined weight of the amount of gelatin and amino acid in the excipient mixture.

The present invention provides for a method of spray drying wherein the feed solution was prepared in water with 6 mg/mL total excipient content and 1% v/v phage stock (˜1010-1011 PFU/ml). The solution was sprayed at 2 mL/min and dried using hot air at an inlet temperature of 60° C. (outlet temperature is ˜36° C.). Spray dried powders were enclosed in glass vials at relative humidity (RH) 30% and stored at −80° C. until analysis. The change in phage titer during spray drying was calculated using feed solution and powder titers. Both titer values were converted to PFU/mg units for use in this calculation. Feed titer (PFU/mg)=Feed titer (PFU/mL)*6.

It is anticipated that the precise settings for spray drying can vary according to external environmental factors and conditions, and that the measured result of the method of the invention can be determined by the yield of product as shown by storage stability, aerosol performance, FPF value, and general % recovery. The methods of the invention provide for formulations which show less than about 1.5 log reduction in phage titer relative to their feed solutions. The methods of the invention provide for high FPF value, of about 74 to 80%. However the in vitro aerosol performance as shown by the ED values of HG-Leu and HG-Trileu formulations were significantly higher than that of HG (p<0.05) (FIG. 9). Moreover, the FPF values of HG90-Leu10, HG95-Leu5 and HG90-Trileu10 formulations were significantly higher (p<0.05) than that of HG; FPF values of the HG95-Trileu5 and HG formulations were similar to each other.

Dispersion aid types: Spray dried particles with enhanced dispersibility can be obtained including leucine and trileucine. Besides, other dispersion enhancers may be used such as beta-cyclodextrin, and hydroxypropyl-beta-cyclodextrin.

The present disclosure provides for the process described above, where the gelatin is unhydrolyzed gelatin, hydrolyzed gelatin or other type of gelatin. In particular the present disclosure provides for the method described above where the gelatin is selected from the group consisting of gelatin, unhydrolyzed gelatin, hydrolyzed gelatin or gelatin analog. The present disclosure provides for the use of gelatin analogs such as semi-synthetic gelatin analogs (gelatin mehacryloyl), agar-agar, carrageenan, alginates, cornstarch, pectin, Hypromellose and the like.

Gelatin: May be bovine, porcine, or fish-source. The gelatin should be easy to dissolve in water and have low viscosity, so they can be spray drying via aqueous solution. The dissolved gelatin should be able to uniformly incorporate other components like phage and dispersion enhancers. Hydrolyzed gelatin or collagen hydrolysate is a more readily soluble form of gelatin and is suitable. Another kind of gelatin that might be suitable is Type B gelatin, which is obtained via alkali hydrolysis of animal tissue and is commonly used as a gelling agent for oral and topical formulations may be suitable.

Thus the present disclosure provides for a method of making a pharmaceutical formulation for administering bacteriophage therapy to a patient in need thereof, comprising:
a. dissolving amino acid and gelatin, or a pharmaceutically acceptable salt thereof, in an aqueous or an organic medium to prepare a solution;
b. optionally adding one or more pharmaceutically acceptable excipients to said solution; and
c. combining bacteriophage suspension to the said solution; and
d. spray-drying to afford said powder composition with an improved inhalable fraction of said bacteriophage and an increased stability and aerosolization stability as compared with bacteriophage alone.

The disclosure provides for the process for making a pharmaceutical formulation as described above where the amino acid is leucine or a derivative, analog or mimetic thereof, such as L-leucine, iso-leucine, tri-leucine or other leucine type of amino acid. Thus the present disclosure provides for a method of manufacture as described above where the amino acid is selected from the group consisting of Leucine, L-leucine, iso-leucine, tri-leucine or leucine analog. In a preferred embodiment the amino acid is Leucine.

The present disclosure provides for the process described above, where the gelatin is unhydrolyzed gelatin, hydrolyzed gelatin or other type of gelatin. In particular the present disclosure provides for the method described above where the gelatin is selected from the group consisting of gelatin, unhydrolyzed gelatin, hydrolyzed gelatin or gelatin analog. The present disclosure provides for the use of gelatin analogs such as semi-synthetic gelatin analogs (gelatin mehacryloyl), agar-agar, carrageenan, alginates, cornstarch, pectin, Hypromellose and the like.

The present disclosure therefor provides for a powdered composition which comprises bacteriophage, gelatin and an amino acid. The present disclosure further provides for where the powder is further formulated into a pharmaceutical composition. The present disclosure also provides for where the pharmaceutical formulation is made directly and optionally incorporates further pharmaceutically acceptable carrier, excipient or diluent.

The present disclosure encompasses the treatment of a patient in need of therapeutic intervention with bacteriophage, comprising administration of a composition of the present disclosure to a patient in need thereof.

The present disclosure thus encompasses the use of the compositions of the disclosure in the treatment of a patient in need of therapeutic administration of bacteriophage.

The present disclosure encompasses therapeutic treatment methods where the bacteriophage compositions of the disclosure are used in combination with other therapeutic treatments.

In particular, the present disclosure provides for a method of treatment of a patient with a bacterial infection, comprising administering a therapeutically effective amount of a pharmaceutical composition as described above to the patient.

The present disclosure provides for the administration of the therapeutic formulations via all medically appropriate routes of administration. In particular, the compositions of the present disclosure demonstrate improved inhalable fraction of bacteriophage. Further, the compositions of the present disclosure demonstrate an improved inhalable fraction of bacteriophage with an increased stability and aerosolization stability as compared with preparations of bacteriophage alone.

In a specific embodiment of the present invention, the methods encompass the development of Pseudomonas aeruginosa bacteriophage 95 as a stable and aerosolizable dry powder formulation.

Thus the present invention encompasses methods for creating a composite spray dried matrix of hydrolyzed gelatin with leucine or trileucine which provides both high bacteriophage stability, and excellent aerosol performance as a dry powder formulation for treating bacterial infection. In a preferred embodiment the bacteriophage is a Pseudomonas aeruginosa bacteriophage.

### Materials and Methods

Bacteriophage and host bacteria: Pseudomonas aeruginosa phage 95 (ATCC 14211-B1) and its host bacteria Pseudomonas aeruginosa (ATCC 14211) were obtained from American Type Culture Collection (ATCC). The bacterial strain is resistant to multiple antibiotics, and it is susceptible to infection by the phage. The bacteria were cultured aerobically in Brain Heart Infusion media broth at 37° C. with shaking at 100 RPM. High-titer phage stocks were prepared using the procedure recommended by ATCC.

### Materials

The excipients used for dry powder formulations were derived from varied sources. D (+) trehalose dihydrate (laboratory grade) was obtained from Spectrum Chemical Mfg. Corp. (New Brunswick, NJ); lactose monohydrate (N.F. grade) was obtained from Avantor, Inc. (Radnor, PA); D-mannitol (R&D grade); gelatin (bovine skin, Type B, Reagent grade) and hydrolyzed gelatin (enzymatic, laboratory grade) were obtained from Sigma-Aldrich (St. Louis, MO). Bacteriological agar was obtained from Thermo Scientific (Waltham, MA) and Brain Heart Infusion culture medium was obtained from Becton, Dickinson and Company (Sparks, MD).

### Culture and Host Bacterium

To obtain a bacterial culture, a frozen stock was streaked onto an agar plate prepared using Brain Heart Infusion (BHI) medium. After incubating the plate at 37° C. for 18-24 h, a bacterial colony was transferred to an Erlenmeyer flask (500 ml) containing 100 ml of BHI broth. An actively growing culture was obtained by incubating the mixture at 37.° C. and 100 RPM shaking for 18-24 h.

### Preparation of Phage Stock

A high-titer suspension of active phages, called phage stock, was prepared using the procedure recommended by ATCC. An overnight broth culture of the host bacterium Pseudomonas aeruginosa was obtained. In a Petri dish, an underlay agar media was set using BHI+1.5% w/v agar. A phage stock obtained from ATCC, or an existing phage stock (a master stock) was diluted to obtain a suspension with an active titer of 107-108 PFU/ml. Then, a melted suspension of BHI+0.5% w/v agar was mixed with 5% v/v overnight bacterial culture and 10% v/v diluted phage suspension. The resulting mixture was poured over the underlying agar media and allowed to solidify for 15 min. The plate was incubated at 37° C. for 18 h. Phage would infect and lyse the bacteria in the overlay mixture, resulting in amplification of the phage titer. To isolate the phages, the overlay agar from each plate was scraped off and mixed with 10 ml BHI medium. The mixture was centrifuged at 1000*g to remove the cell debris and agar. The supernatant was filtered using a 0.22 μm syringe filter (non-protein binding) to remove traces of bacteria. The filtered phage stock was stored at 4° C.

Phage titer analysis: Active phage titer in a formulation was quantified using a double agar plaque assay as described in literature with some modifications (Kropinski et al. 2007). It is briefly described as follows. A suspension of known concentration (mg/mL) is prepared for a sample and serially diluted. An overnight broth culture of the host bacteria Pseudomonas aeruginosa is obtained. In a petri dish, an underlay agar media is set using 1.5% agar. Then, a “softer” overlay agar medium is prepared using 0.5% agar. The melted overlay medium (at 40° C.) is mixed with 5% v/v bacterial culture before setting it above the underlay agar. Once set, the diluted phage samples are added as ten μL spots over the overlay agar. Thus prepared, the petri dish is covered and incubated at 37° C. for 18 hours. Each active phage in the spot would lyse bacterial cells present in the overlay agar medium, which over time results in a visible zone of lysis called a plaque. The spot containing 10-40 plaques is preferred for counting. By multiplying the number of plaques with the dilution factor, the phage titer of the original sample can be calculated. Thus, the unit of phage titer is plaque forming units per mL (PFU/ml).

Spray drying: Spray dried formulations were prepared using a BUCHI B-290 spray dryer in open-loop configuration. The feed solution was prepared in water with 6 mg/mL total solid content and 1% v/v phage stock (˜10¹⁰-10¹¹ PFU/ml). The solution was sprayed at 2 mL/min and dried using hot air at 60° C. The spray dried powders were enclosed in glass vials at RH<30% and stored at −80° C. until analysis. The change in phage titer during spray drying was calculated using feed solution titer and powder product titer. Both titer values were converted to PFU/mg unit for this calculation. Feed titer (PFU/mg)=Feed titer (PFU/mL)*6.

Change in spray drying titer=log (SD powder titer)−log (Feed solution titer)

### Glass Transition Temperature

The thermograms of the spray dried excipients were measured using a Discovery DSC 2500 (TA Instruments, New Castle, DE), a differential scanning calorimeter. Spray dried phage formulations were prepared and immediately sampled for DSC analysis. About 3-5 mg of powder was loaded into a Tzero aluminum pan (TA Instruments, New Castle, DE, USA) and hermetically sealed with a lid containing a pinhole. To begin the analysis, the powder was cooled to 0° C. and stabilized for 5 min. Then, the sample pan was heated to 250° C. at 2° C./min with a modulation of 1° C./min. Thus, the reversible and irreversible heat flow rate profiles were obtained. The reversible heat flow profile was observed for a step-change in the baseline, which indicates a glass transition. The reported glass transition temperature corresponded to the midpoint value of this step change, which was analyzed using the TRIOS analytical software.

Invitro aerosol performance: A Multi-Stage Liquid Impinger (MSLI) (Copley Scientific, Nottingham, UK) was used to simulate and quantify inhalation performance of spray dried formulations. The formulation was loaded in HPMC size-3 capsules (Qualicaps, Whitsett, NC) and actuated through a low-resistance RS01 DPI device (Plastiape S.p.A., Osnago, Italy) into the MSLI assembly. During actuation, four liters of air was passed through the device at 86 L/min, which corresponds to 4 kPa suction pressure. The dispersed particles were collected from capsule, device, mouthpiece adaptor, USP inlet, four MSLI stages, and exhaust filter using BHI culture medium. The viable phage titer deposited in each region was quantified using plaque assay. The dispersion of each formulation was tested using three replications of 20 mg each. The percentage of the initial phage in the capsule that is recovered from all regions after analysis is termed the % recovery. The percentage of phage that is recovered downstream of the device is termed emitted dose (ED). Fine particle dose (FPF) is the percentage of the phage contained in particles with aerodynamic diameter below 5 μm.

Storage stability: Powder samples were sealed in glass vials at a low ambient RH (<30%) and stored at 4° C. The phage titer of the formulation was measured after 2, 4, and 12 weeks.

Powder X-ray diffraction: A Rigaku Smartlab™ diffractometer (Rigaku Americas, The Woodlands, TX) with a Cu-Kα radiation source and a D/tex ultra detector was used to obtain X-ray diffraction pattern of formulations. X-ray source was operated at 40 kV voltage and 44 mA current. The diffraction patterns were recorded at 2θ=4° to 40° and at a scan speed of 4°/min.

Particle size: Particle size was determined using a Malvern Mastersizer 3000 coupled to an Aero-S for dry powder dispersion (Malvern Instruments, Worcestershire, UK). Sample feed rate was set to 100%. Compressed air pressure of 4 bars was used for powder carriage and dispersion through the optical cell. Volume percentile diameters D₁₀, D₅₀, and D₉₀ were used to characterize the particle size distribution of the powders. The broadness of the distribution was using Span, which is a ratio of (D₉₀−D₁₀) to D₅₀.

Particle morphology: Formulation particles were examined using a scanning electron microscope. NOVA nanoSEM (FEI Company, Hillsboro, Oregon, USA) was used for Part 1 of the study and Apreo 2 S (Thermo Fisher Scientific, Waltham, Massachusetts, USA) was used for Part 2. The powder sample was mounted on a sample stub via an adhesive carbon tape. The particles were coated with a thin platinum film by sputtering at 40 mA for 1 min (208 HR, Cressington Sputter Coater, England, UK). The coated particles were imaged at 5 kV acceleration voltage at various magnifications.

### Statistical Analysis

One-way ANOVA was used to statistically compare three or more formulations (α=0.05). Tukey's honestly significant difference test was used as the post hoc test to perform pairwise comparison of 3 or more formulations.

### Results

### Part 1: Impact of Protective Excipients for Phage Stability and Aerosol Performance

### Phage Stabilization During Spray Drying

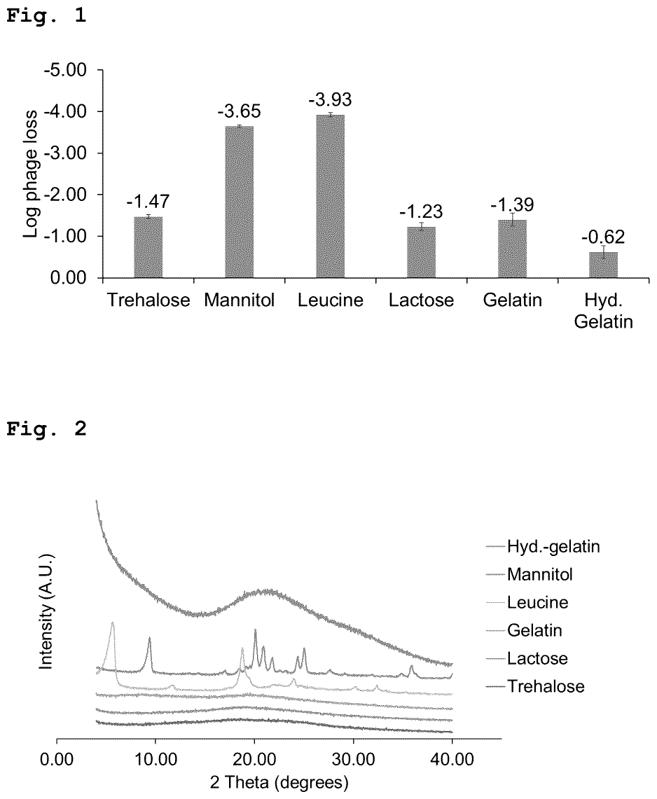

Titer loss of Pa 95 phage due to spray drying was determined for single-excipient phage formulations (FIG. 1). Lactose, trehalose, gelatin, and hydrolyzed gelatin were good stabilizers of phage, showing less than 1.5 log reduction of Pa 95 phage titer relative to their feed solutions. Spray dried hydrolyzed gelatin showed significantly lower phage titer loss than other formulations. On the other hand, mannitol and leucine showed >3.5 log loss of phage titer due to spray drying. Powder X-ray diffraction analysis showed that spray dried phage formulations of lactose, trehalose, gelatin and hydrolyzed gelatin showed negligible crystallization while those of mannitol and leucine were highly crystalline (FIG. 2). Thus, phage stabilization was correlated to the solid state of the excipient matrix: amorphous matrix of sugars and gelatin preserved phage viability while the crystalline matrix of mannitol and leucine showed low phage viability.

The titer change of Pseudomonas aeruginosa phage 95 phage during spray drying is illustrated in FIG. 1. The formulations with lactose, trehalose, gelatin, and hydrolyzed gelatin (HG) showed less than 1.5 log reduction of phage titer relative to their feed solutions indicating better stability than mannitol and leucine, which showed more than 3.5 log reduction. In particular, the spray dried formulation with hydrolyzed gelatin showed significantly smaller phage titer reduction than those with trehalose, lactose, and gelatin (p<0.05) formulations.

Powder X-ray diffraction analysis showed that spray dried phage formulations with lactose, trehalose, gelatin, or hydrolyzed gelatin showed no apparent crystalline peaks; while those with mannitol, or leucine showed clear crystalline peaks.

### Particle Size and Morphology

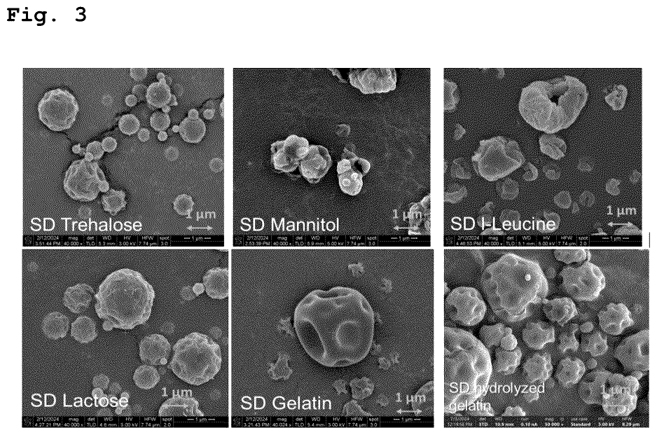

Particle size analysis showed that all spray dried formulations showed inhalable size range (Table 1). Particle morphology after spray drying is shown in FIG. 3. Spray dried trehalose, lactose, gelatin and hydrolysed gelatin particles were round particles with dimples and uneven surfaces. Mannitol and leucine particles were irregular and broken. No agglomeration was observed in any formulation.

Particle size analysis showed that all spray dried formulations had a fine size range (<5 μm) (Table 1). The particle morphology after spray drying is demonstrated in FIG. 3. The particles spray dried with either mannitol or leucine have irregular shape. The other spray dried formulations exhibited round particles with corrugated surfaces.

TABLE 1
Percentile particle size of spray dried formulations. Data is
shown as mean ± standard deviation (n = 4).

| Formulation | D₁₀ (μm) | D₅₀ (μm) | D₉₀ (μm) | Span |
|---|---|---|---|---|
| Trehalose | 0.39 ± 0.03 | 1.28 ± 0.03 | 3.25 ± 0.17 | 2.24 |
| Mannitol | 1.40 ± 0.02 | 2.89 ± 0.06 | 6.05 ± 0.19 | 1.61 |
| Leucine | 0.94 ± 0.01 | 1.86 ± 0.03 | 4.42 ± 0.16 | 1.87 |
| Lactose | 0.63 ± 0.01 | 1.42 ± 0.01 | 2.89 ± 0.06 | 1.59 |
| Gelatin | 0.87 ± 0.02 | 2.17 ± 0.05 | 4.86 ± 0.06 | 1.84 |
| Hydrolysed | 0.59 ± 0.03 | 1.43 ± 0.11 | 4.60 ± 2.51 | 2.76 |
| gelatin | | | | |

### Invitro Aerosol Performance

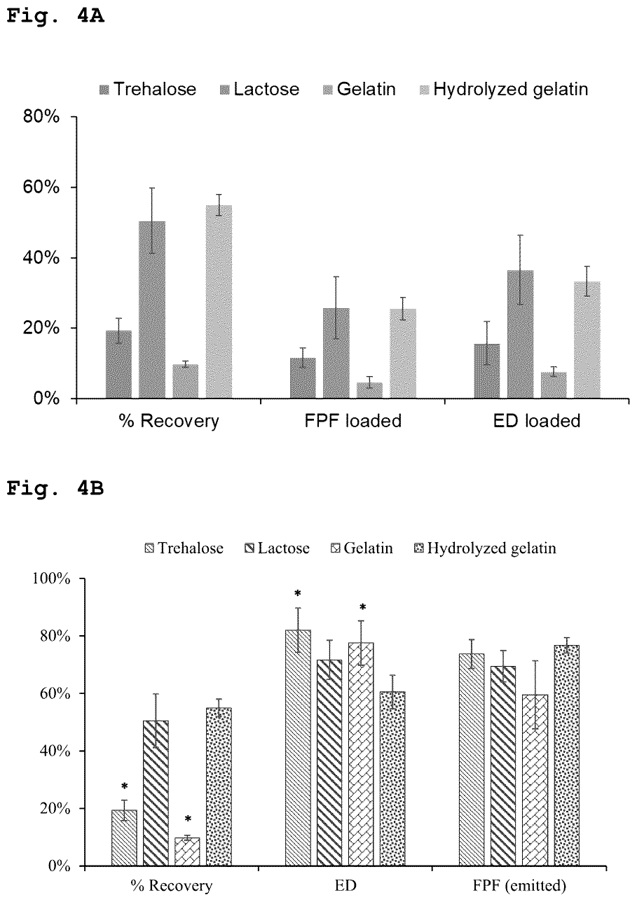

It was observed that phage recovery was low and variable among different phage formulations (FIG. 4): lactose (50%) and hydrolyzed gelatin (55%) showed higher recovery than trehalose (19%) and gelatin (10%). The low recovery of active phage could be due to inactivation of phage during dispersion testing. ED and FPF were directly affected by low phage recovery.

Lactose and hydrolysed gelatin formulation showed higher ED (˜35%) and FPF (26%) of viable phage. Gelatin and trehalose formulation showed poor ED and FPF (FIG. 4).

No significant difference (p<0.05) was observed in the emitted dose among the formulations with trehalose, lactose and gelatin; the formulation with hydrolyzed gelatin showed a lower emitted dose of 60±6% (FIG. 4). The FPF values of the spray dried formulations, from highest to lowest, were 77±3% for hydrolyzed gelatin, 74±5% for trehalose, 69±5% for lactose and 59±12% for gelatin. Thus, hydrolyzed gelatin formulation showed the highest FPF. Based on ED and FPF, all formulations exhibited good aerosol performance.

### Shelf Stability

Stability of phage titer in spray dried gelatin and hydrolyzed gelatin was excellent during storage at 4° C. in closed vials, showing negligible change in phage titer over 12 weeks (FIG. 5). The titer reduced slightly (˜0.5 log) for trehalose and lactose formulations after 4 weeks but did not show further reduction. Thus, gelatin, hydrolyzed gelatin, trehalose, and lactose maintained acceptable titer of Pa 95 phage in spray dried formulations for 12 weeks.

### Glass Transition Temperature

Gelatin and hydrolyzed gelatin showed significantly higher glass transition temperatures (Tg) than trehalose and lactose as shown in the table of FIG. 10.

### Part 2: Enhancing the Aerosol Performance

Based on the phage retention and stability results for different excipients, we determined hydrolyzed gelatin to be the most effective stabilizer. We studied this formulation further to improve the aerosol performance. The effect of adding dispersion enhancers like leucine and trileucine to HG on aerosol performance was studied. We compared two proportions for each: 5% and 10% w/w. Thus, we studied four formulations: HG90Leu10, HG90Trileu10, HG95Leu5 and HG95Trileu5.

Based on the phage titer and stability results, hydrolyzed gelatin exhibited promising phage stabilization in a spray dried formulation. Therefore, we further studied the impact of incorporating dispersion enhancers like leucine and trileucine into the hydrolyzed gelatin formulations with the aim of improving aerosol performance. We prepared four formulations with 5% or 10% w/w of either leucine or trileucine added to hydrolyzed gelatin, as shown in the table of FIG. 11. The formulation with only hydrolyzed gelatin was used as control and marked as HG. All formulations were produced by spray drying using the same operation parameters listed above.

Phage Stability after Spray Drying

Titer loss (log) value of formulations with 5% leucine, 5% trileucine and 10% trileucine was around −0.70, which resembles that of 100% HG formulation (FIG. 6). However, the titer loss was slightly higher for 10% leucine (−1.24 log). Overall, spray dried HG-leucine and HG-trileucine stabilize phage well.

### Solid-State and Particle Properties

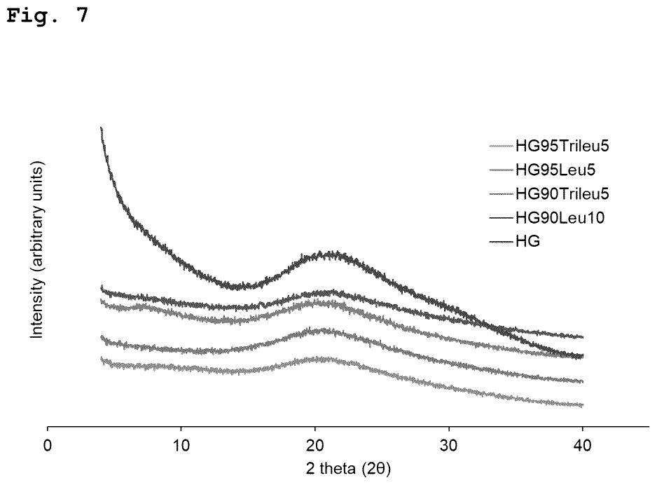

X-ray diffraction patterns showed that all leucine and trileucine formulations were amorphous right after preparation (FIG. 7). For all leucine and trileucine formulations, a major amount of particles had sizes between 0.5 and 5.0 micron in size (Table 2). This size range is favorable for inhalation.

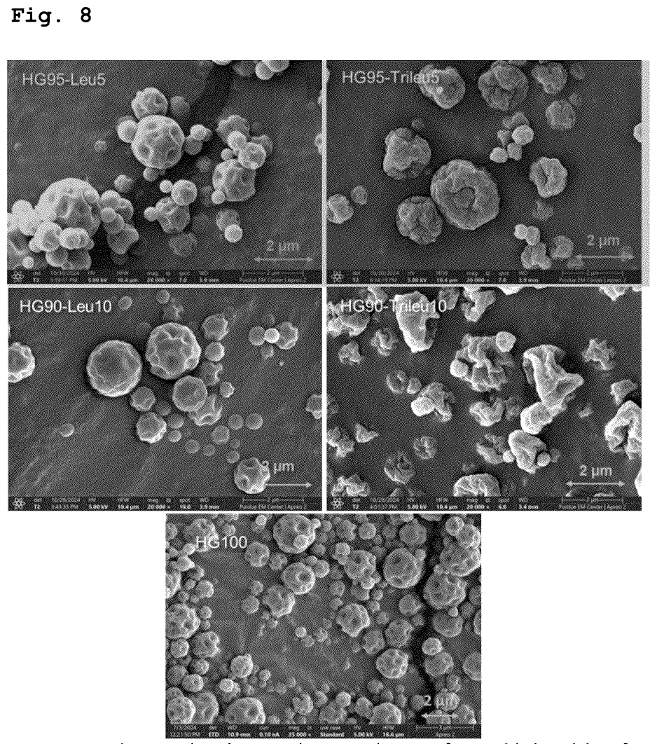

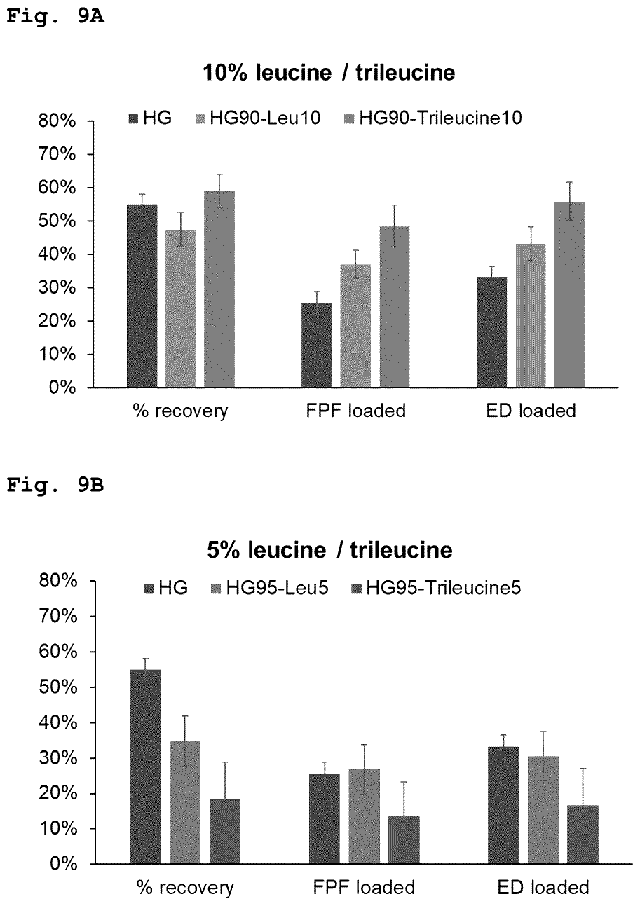

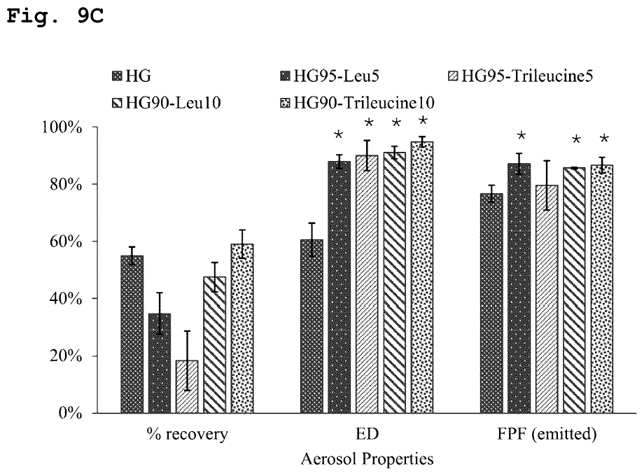

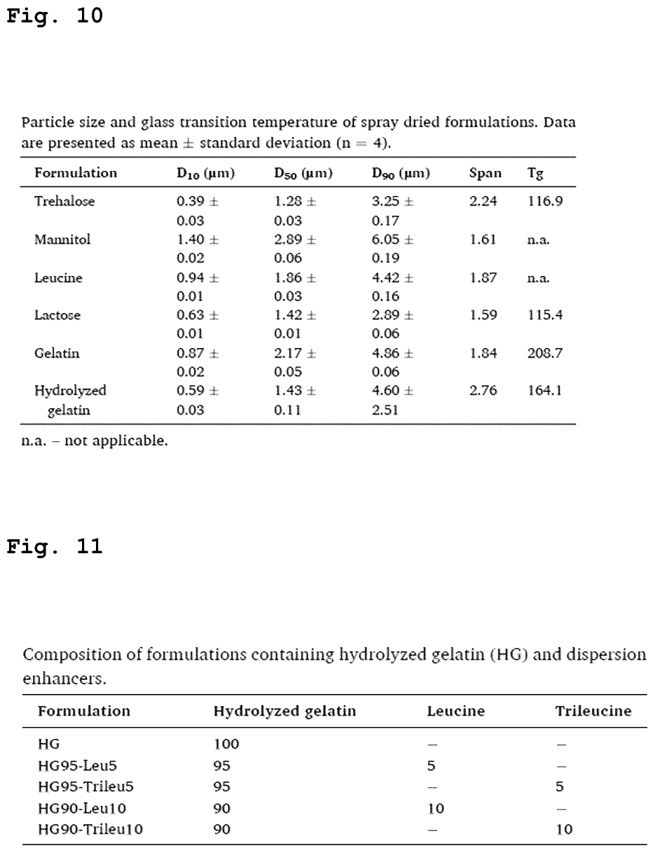

SEM imaging showed that leucine-containing formulations were round with smooth surface and large dimple-like depressions (FIG. 8). In contrast, trileucine-containing particles were crumpled with highly rough surfaces. The 5% trileucine particles were mostly globular while the 10% trileucine particles were highly deformed. None of the formulations show particle fusion or agglomeration.

TABLE 2
Percentile particle size of spray dried HG-leucine and
HG-trileucine formulations. Data is shown as mean ±
standard deviation (n = 4-5).

| Formulation | D₁₀ (μm) | D₅₀ (μm) | D₉₀ (μm) | Span |
|---|---|---|---|---|
| HG90Leu10 | 0.69 ± 0.05 | 1.49 ± 0.36 | 3.09 ± 0.27 | 1.608 |
| HG90Trileu10 | 0.767 ± 0.01 | 1.54 ± 0.01 | 2.94 ± 0.04 | 1.408 |
| HG95Leu5 | 0.652 ± 0.05 | 1.42 ± 0.36 | 2.83 ± 0.08 | 1.533 |
| HG95Trileu5 | 0.717 ± 0.01 | 1.53 ± 0.06 | 3.38 ± 0.52 | 1.738 |
| HG100 | 0.59 ± 0.03 | 1.43 ± 0.11 | 4.60 ± 2.51 | 2.756 |

### Invitro Aerosol Performance

The ED values of HG-Leu and HG-Trileu formulations were significantly higher than that of HG (p<0.05) (FIG. 9). Moreover, the FPF values of HG90-Leu10, HG95-Leu5 and HG90-Trileu10 formulations were significantly higher (p<0.05) than that of HG; FPF values of the HG95-Trileu5 and HG formulations were similar to each other.

Aerosol performance of formulations with 10% leucine/trileucine was significantly better than HG (FIG. 9); ED and FPF for 10% trileucine (56% and 49%) were slightly better than those for 10% leucine (43% and 37%). In comparison, HG formulation without leucine or trileucine showed 33% ED and 26% FPF. Interestingly, the comparison of MSLI dispersion results of HG, 10% leucine and 10% trileucine formulations is rendered simpler due to similar phage recovery.

On the other hand, HG-phage formulations with 5% leucine or 5% trileucine did not show significant improvement in ED or FPF (viable phages) compared to HG-phage formulation (FIG. 9). This may be attributed to a lower recovery of viable phage in these formulations than in the HG-phage formulation. A higher rate of phage inactivation or denaturation may have reduced phage recovery in 5% leucine/trileucine formulations during dispersion tests.

Thus, spray dried 90:10 HG-leucine and 90:10 HG-trileucine matrix are not only good phage stabilizers but are also more suitable for pulmonary delivery than 100% HG matrix.

### CONCLUSIONS

### Part 1

Gelatin and hydrolyzed gelatin were highly suitable for phage stabilization during spray drying and storage. Trehalose and lactose were also good stabilizers, showing only a slight reduction in titer during storage. Phage stabilization correlated to the solid state of the stabilizers: amorphous matrix of trehalose, lactose and the gelatins imparted good stability while the crystalline matrix of leucine and mannitol was a poor stabilizer. Spray dried lactose and hydrolyzed gelatin powders showed good invitro aerosol performance, which is conducive to their pulmonary delivery. Phage recovery after MSLI tests for trehalose and gelatin were low, which caused a poor lung dose of active phage.

For an effective phage formulation for inhalation, it is important to have a high viable phage titer after preparation and storage. It is equally critical for the powder to efficiently deliver the viable phages to the lungs. Based on these, we determined hydrolyzed gelatin as the most effective spray drying excipient for a dry powder formulation of the Pseudomonas phage.

### Part 2

Leucine and trileucine were added to HG formulation as dispersion enhancers to improve powder dispersion properties. Phage preservation capacity remained unaffected with the addition of leucine and trileucine at 5 and 10% to hydrolyzed gelatin. However, 10% leucine showed a slightly higher phage loss than others. Like the pure HG matrix, the amorphous state of leucine-HG and trileucine-HG formulations could have favored phage stability. Compared to pure HG formulation, 10% leucine and 10% trileucine significantly improve the aerosol performance of HG particles, with the latter showing higher emitted phage dose and fine particle dose. However, the aerosol properties of 5% leucine and trileucine formulations were similar to that of HG. This was partly attributed to phage inactivation during powder dispersion, which resulted in a lower overall phage viability in 5% leucine/trileucine formulation than HG formulation.

Dispersion aid composition in the mix 10-20% w/w or more may be suitable. Phage stabilizing formulation may be generated with gelatin without using a dispersion agent. However, the aerosol performance of these would be expected to be lower. The composition should offer acceptable phage stabilization (<1.0 log change) as well as good aerosolization properties to the dry powder. Thus the present methods and formulation were prepared.

Among the four formulations, spray dried 90:10 HG-trileucine was the most phage-stabilizing and dispersion-enhancing powder matrix for the Pseudomonas phage.

Dispersion aid composition in the mix 10-20% w/w or more may be suitable. Phage stabilizing formulation may be generated with gelatin without using a dispersion agent. However, the aerosol performance of these could be lower. The composition should offer acceptable phage stabilization (<1.0 log change) as well as good aerosolization properties to the dry powder.

Bacteriophages are highly promising antibacterial drug candidates in the face of rapidly expanding antibiotic resistance in pathogenic Gram-negative bacteria. To target bacterial infections of the lungs, delivery of bacteriophage through the pulmonary route or inhalation would result in a higher local phage concentration than delivery through the intravenous or oral route. Inhalable dry powder formulations are highly suitable for this due to their ease of administration, large dose capacity and superior storage stability. However, bacteriophages (or phages) are highly labile biological entities that can be easily inactivated by chemical and thermal treatments involved in the preparation, storage and use. Thus, it is essential to include excipients in formulations that can preserve phage viability. Therefore, the purpose of this study was to analyze the effect of various pulmonary excipients on phage stability in dry powder formulations prepared using spray drying. Pseudomonas aeruginosa phage 95 was used as the model phage for this study.

The stability of phage formulation correlated with the solid state of the excipient matrix: amorphous matrix of trehalose, lactose and gelatin preserved phage well while the crystalline matrix of mannitol and leucine showed poor phage viability. The ability of the amorphous matrix of trehalose and lactose to stabilize bacteriophages has been reported in numerous studies. The amorphous solid forms of trehalose and lactose have a relatively high glass transition temperatures, which suggests a low molecular mobility at ambient temperatures. This would immobilize the native structure of a phage, which can prevent denaturation and inactivation of the phage in the absence of water. It is likely that the amorphous form of the spray dried gelatin and hydrolyzed gelatin preserved phage due to a similar mechanism. On the other hand, crystalline solid matrices cannot form a preservative matrix around the phage particle due to phase separation. Thus, phage titer was significantly reduced in the spray dried leucine and mannitol.

The change in phage titer due to spray drying in the hydrolyzed gelatin formulation was significantly lower than that for the trehalose, lactose, and gelatin formulations. In addition, the spray dried gelatin and hydrolyzed gelatin formulations exhibited better phage stability than the spray dried trehalose and lactose formulations during the storage at 4° C. This could be related to the observation of higher glass transition temperatures (Tg) of gelatin and hydrolyzed gelatin compared to trehalose and lactose. Glassy amorphous matrices with higher Tg would have a lower degree of molecular motion at a given temperature less than Tg, which would reduce phage denaturation due to limit molecular motion.

The hydrolyzed gelatin formulation showed higher FPF values than the trehalose, lactose and gelatin formulations. Based on the emitted dose and fine particle fraction values, all stabilizing excipients-hydrolyzed gelatin, gelatin, trehalose and lactose-showed good aerosol performance. Interestingly, phage recovery after aerosol testing varied with the type of stabilizer (FIG. 4); lactose (50%) and hydrolyzed gelatin (55%) showed significantly higher recovery (p<0.05) than trehalose (19%) and gelatin (10%). We found that the poor phage recovery after powder dispersion was caused by denaturation of phage during the aerosolization process. This has been reported in other studies, wherein this was attributed to impact forces experienced by the phage during dispersion. Such forces can damage phage particles immobilized close to the particle surface. The variation in recovery values between the four excipients might correspond to difference in the fraction of phages close to the particle surface. However, detailed particle surface analysis is warranted to validate this hypothesis in future.

We further studied the impact of incorporating dispersion enhancers such as leucine and trileucine into the spray dried hydrolyzed gelatin (HG) formulation to further improve aerosol performance. The spray dried HG-leucine and HG-trileucine formulations, each at 90:10 and 95:5 ratios, showed similar phage titer retention as the spray dried HG. This could be explained by the fact that HG-leucine and HG-trileucine formulations were also amorphous solids (FIG. 7). Importantly, the addition of leucine and trileucine to hydrolyzed gelatin for spray drying improved aerosol performance, leading to higher emitted dose and fine particle fraction (FIG. 9). As leucine and trileucine accumulate at the droplet surface during spray drying the resultant dried particles would have an outer shell that is rich in leucine/trileucine molecules. The presence of leucine/trileucine on the particle surface reduces surface energy, which translates to improved powder flow and dispersion.

In our study, hydrolyzed gelatin seems a promising phage stabilizer in the dry state. For the first time, we report on the ability of gelatin to stabilize phages in the spray dried powders. Gelatin has been previously used as an inactive ingredient in FDA approved drug formulation for inhalation and therefore can be generally considered safe for inhalation products.

For an effective phage dry powder formulation for inhalation, it is important to maintain a high phage titer after preparation and storage. It is equally critical that the powder efficiently delivers the viable phages to the lungs. Our study demonstrated that hydrolyzed gelatin, used as an excipient, provides superior phage stabilization during spray drying and storage compared to the traditional sugar protectants such as trehalose and lactose. Our work also indicated that the amorphous matrix of trehalose, lactose, hydrolyzed gelatin and gelatin provide better protection for phage stability than the crystalline matrices of leucine and mannitol. All spray-dried phage formulations showed relatively high emitted dose and fine particle fraction values. The addition of leucine and trileucine, up to 10% w/w, into the amorphous hydrolyzed gelatin matrix further improved aerosol performance without compromising phage stability. This is the first study to report that the amorphous matrix of gelatin containing leucine or trileucine offers both superior aerosol performance and satisfactory stability to spray-dried phage formulations intended for inhalation. Thus, these formulations show promise for the treatment of pulmonary bacterial infections. It is worth noting that the phage stability results in gelatin matrix may be phage specific; further studies on other types of phages and on the mechanism by which gelatin stabilizes phages in the dry state are warranted.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

We claim:

1. A method for manufacturing a powder composition consisting of an amino acid, gelatin and bacteriophage, comprising:
c. preparing a bacteriophage suspension;
d. prepare an excipient solution by dissolving an amino acid and gelatin, or a pharmaceutically acceptable salts thereof, in an aqueous or an organic solvent;
c. create a mixture by combining bacteriophage suspension to the excipient solution; and
d. spray-drying the mixture to create a powder composition.

2. The method of claim 1, where the amino acid is selected from the group consisting of nonpolar, non aromatic amino acids glycine, alanine, valine, leucine and isoleucine.

3. The method of claim 2 where the amino acid is leucine or a derivative, analog or mimetic thereof.

4. The method of claim 3 where the leucine or derivative, analog or mimetic thereof is selected from the group L-leucine, iso-leucine, or tri-leucine.

5. The method of claim 1 where the gelatin is a unhydrolyzed gelatin, hydrolyzed gelatin or other type of gelatin or gelatin analog.

6. The method of claim 1 which results in a bacteriophage-excipient powder composition that provides for an inhalable fraction of bacteriophage with an increased stability and aerosolization stability as compared with bacteriophage alone.

7. The method of claim 7 where the gelatin analog is semi-synthetic gelatin analogs (gelatin mehacryloyl), agar-agar, carrageenan, alginates, cornstarch, pectin, Hypromellose and the like.

8. The method of claim 1 where the bacteriophage targets a bacteria which infects mammals.

9. A method for making a pharmaceutical formulation of bacteriophage comprising
a. dissolving amino acid and gelatin, or a pharmaceutically acceptable salt thereof, in an aqueous or an organic medium to prepare an excipient solution;
b. optionally adding one or more pharmaceutically acceptable excipients, carrier or diluent to the excipient solution;
c. preparing a bacteriophage suspension;
c. combining the bacteriophage suspension with the excipient solution to make a mixture; and
d. spray-drying the mixture to create a powder composition.

10. The method of claim 9, which results in a dried powder composition which contains bacteriophage where said powder composition has an improved inhalable fraction of bacteriophage and an increased stability and aerosolization stability as compared with bacteriophage alone.

11. The method of claim 9 where the gelatin is selected from the group consisting of unhydrolyzed gelatin, hydrolyzed gelatin or other type of gelatin or gelatin analog.

12. The method of claim 9 where the amino acid is selected from the group consisting of Leucine, L-leucine, iso-leucine, or tri-leucine

13. A bacteriophage powder composition made by the method of claim 1.

14. A bacteriophage pharmaceutical composition made by the method of claim 9.

15. The use of a composition of claim 14 for the treatment of a bacterial disease in a patient in need thereof.

16. The use of a composition of claim 12 for the treatment of a bacterial disease in a patient in need thereof.

17. A method for treating or prophylactically treating a patient with a bacterial infection comprising administering a therapeutic amount of a composition of claim 13 to a patient in need thereof.

18. The method of claim 15 where administration is in combination with or coordination with another therapeutic agent.

19. The method of claim 16 where the administration of the therapeutic dose is via any medically appropriate route of administration.

20. A method for treating a mammal for bacterial infection, said method comprising administering a therapeutic amount of a pharmaceutical composition comprising a powder composition consisting of an amino acid, gelatin and bacteriophage.
